(19) United States
(12) Patent Application Publication
(10) Pub. No.: US 2022/0215964 A1
(43) Pub. Date: Jul. 7, 2022

(54) APPARATUS AND METHOD FOR PROVIDING HEALTH ANALYSIS SERVICE

(71) Applicant: Hyung Woon KIM, Seongnam-si (KR)

(72) Inventor: Hyung Woon KIM, Seongnam-si (KR)

(21) Appl. No.: 17/600,586

(22) PCT Filed: May 14, 2020
(86) PCT No.: PCT/KR2020/006363

§ 371 Date: Sep. 30, 2021

(30) Foreign Application Priority Data
May 14, 2019 (KR) 10-2019-0056438

(51) Int. Cl. G16H 50/30; G16H 20/90; G16H 10/60

(52) U.S. Cl. CPC G16H 50/30; G16H 10/60; G16H 20/90

(57) ABSTRACT

The present invention relates to an apparatus and method for providing a health analysis service, the apparatus comprising: a communication circuit for communicating with a user terminal; and a processor for controlling the communication circuit to perform a health analysis on the basis of user information received through the communication circuit and transmit a health analysis result to the user terminal. The processor analyzes the natural physical constitution of a user by using the user's date and time of birth and the address of the user's birthplace included in the user information, and predicts a possible disease in accordance with the natural physical constitution and the timing of onset of the possible disease.

### FIELD OF INVENTION

The present invention relates to an apparatus and a method of providing a heath analysis service, and more particularly to an apparatus and a method of providing a heath analysis service for predicting a disease and the onset of possible disease based on inborn body characteristics.

### BACKGROUND OF INVENTION

Recently, as interest in health increases, research has been conducted into technology for systematic health management and disease prevention. In particular, recently, as portable electronic devices such as smartphones and tablet PCs, which are capable of communicating, have become widely popular, services for managing personal health of a user from a remote location are increasingly used.

Health care services to date have largely focused on measurement of the current health status of a user. Although a method of predicting future health is developed in some researches, most methods of predicting future health are based on past statistics, and thus, are uniform and do not attract interest of the user.

### SUMMARY OF INVENTION

### Technical Problem to be Solved

The present invention provides an apparatus and a method of providing a service for providing health-related information customized to the user.

The present invention provides an apparatus and a method of providing a health analysis service for analyzing a vulnerable part of the body and a possible disease based thereon based on inborn body characteristics of a user and predicting the onset of the possible disease.

### Technical Solution

Therefore, the present invention provides a health analysis service provision apparatus including a communication circuit configured to communicate with a user terminal, and a processor configured to perform health analysis based on user information received through the communication circuit and to control the communication circuit to transmit a health analysis result to the user terminal, wherein the processor analyzes inborn body characteristics of a user using a date of birth of the user and a birth address, included in the user information, and predicts a possible disease and an onset of the possible disease based on the inborn body characteristics of the user.

The processor may generate zodiac signs at a time of birth corresponding to the date of birth and the birth address, may detect at least one planet placed at at least one preset angle based on a center of the zodiac signs, and may determine a body part corresponding to the zodiac signs in which the detected planet is placed, as a vulnerable part, to determine the inborn body characteristics.

The at least one preset angle may include at least one of 45 degrees, 90 degrees, and 180 degrees.

The processor may determine the possible disease based on the vulnerable part.

The processor may determine a time when the at least one detected planet is placed in zodiac signs at a time of birth based on ephemeris and may determine the determined time as the onset of the possible disease.

The processor may determine likelihood of the possible disease based on similarity between an angle of the at least one planet detected at an arbitrary time based on the zodiac signs and an angle of the planet placed in the zodiac signs at the time of birth.

Information on the likelihood over time may be transmitted to the user terminal to be output in a form including at least one of a graph, a figure, or a number by the user terminal.

The processor may detect at least one different planet placed at at another preset different angle based on the center of the zodiac signs and may determine a body part corresponding to a constellation in which the at least one detected different planet is placed, as a healthy part of the user.

The at least one preset different angle may include at least one of 30 degrees, 60 degrees, or 120 degrees.

According to an embodiment of the present invention, a method of providing a health analysis service of a server includes receiving user information from a user terminal, analyzing inborn body characteristics of the user using a date of birth of the user and a birth address, included in the user information, and predicting a possible disease and an onset of the possible disease based on the inborn body characteristics.

The analyzing may include generating zodiac signs at a time of birth corresponding to the date of birth and the birth address, detecting at least one planet placed at at least one preset angle based on a center of the zodiac signs, and determining a body part corresponding to the zodiac signs in which the detected planet is placed, as a vulnerable part.

The predicting may further include determining the possible disease based on the vulnerable part, determining a time when the at least one detected planet is placed in zodiac signs at a time of birth based on ephemeris, by the processor, and determining the determined time as the onset of the possible disease.

The predicting may further include determining likelihood of the possible disease based on similarity between an angle of the at least one planet detected at an arbitrary time based on the zodiac signs and an angle of the planet placed in the zodiac signs at the time of birth.

Information on the likelihood over time may be transmitted to the user terminal to be output in a form including at least one of a graph, a figure, or a number by the user terminal.

The analyzing may further include detecting at least one different planet placed at at another preset different angle based on the center of the zodiac signs, and determining a body part corresponding to a constellation in which the at least one detected different planet is placed, as a healthy part of the user.

### Effect of Invention

An apparatus and a method of providing a health analysis service according to the present invention may provide health analysis information customized to a user.

The apparatus and the method of providing a health analysis service according to the present invention may analyze a vulnerable part of the body and a possible disease based thereon and may also provide information on the onset of possible disease to the user, thereby effectively treating and preventing the disease.

### BRIEF DESCRIPTION OF DRAWINGS

FIG. 1 is a diagram showing a health analysis service provision system according to an embodiment of the present invention.

FIG. 2 is a block diagram showing the configuration of a server according to an embodiment of the present invention.

FIG. 3 is a block diagram showing the configuration of a user terminal according to an embodiment of the present invention.

FIG. 4 is a flowchart of a method of providing a health analysis service of a server according to an embodiment of the present invention.

FIG. 5 is a flowchart of a health analysis method of a server according to an embodiment of the present invention.

FIGS. 6 to 8 are diagrams for explaining a health analysis method according to an embodiment of the present invention.

FIG. 9 is a flowchart of a method of providing a health analysis service of a user terminal according to an embodiment of the present invention.

FIG. 10 is a diagram showing an image of a health analysis result output on a user terminal according to an embodiment of the present invention.

FIG. 11 is a flowchart showing a method of providing a health analysis service according to an embodiment of the present invention.

FIG. 12 is a flowchart showing a method of providing a health analysis service according to another embodiment of the present invention.

### BEST MODE

In the description of embodiments of the specification, certain detailed explanations of related art are omitted when it is deemed that they may unnecessarily obscure the essence of the present invention.

The terms “comprises” and “comprising” when used in this specification specify the presence of stated functions, operations, and components, but the presence or addition of one or more other functions, operations, and components is not precluded. It will be further understood that the terms “comprises” or “comprising” when used in this specification, specify the presence of stated features, integers, steps, operations, elements, or components, but do not preclude the presence or addition of one or more other features, integers, steps, operations, elements, components, or groups thereof.

The singular expressions in the present specification include the plural expressions unless clearly specified otherwise in context.

Hereinafter, the present invention will be described with reference to the accompanying drawings.

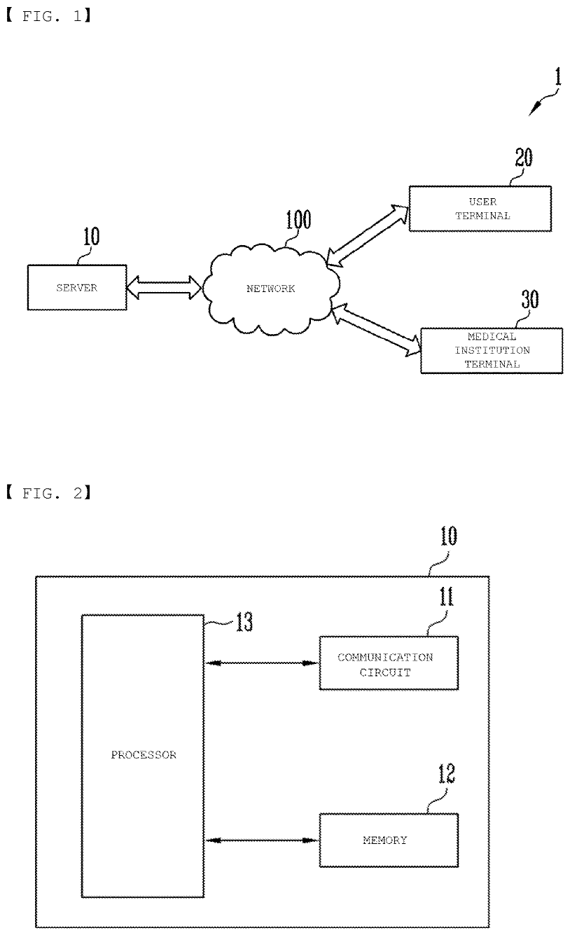

FIG. 1 is a diagram showing a health analysis service provision system according to an embodiment of the present invention.

Referring to FIG. 1, a health analysis service provision system 1 according to an embodiment of the present invention may include a server 10 and a user terminal 20.

The server 10 may communicate with the user terminal 20. For example, the server 10 may receive a request signal to use a health analysis service according to the present invention from the user terminal 20. Upon receiving the request signal, the server 10 may store and manage terminal information for providing the health analysis service to the corresponding user terminal 20. According to an embodiment, the terminal information may include identification information (e.g., device identification (ID)) of the corresponding user terminal 20, and network address information (e.g., a media access control (MAC) address).

According to various embodiments of the present invention, the server 10 may make a request to the user terminal 20 that transmits the request signal for user information and may store and manage the user information received from the user terminal 20. According to an embodiment, the user information may include the date of birth, birth address, genetic test record, etc. of the user of the corresponding user terminal 20.

The server 10 may perform health analysis of the user based on the user information received from the user terminal 20. According to various embodiments of the present invention, the server 10 may analyze the inborn body characteristics of the user from the user information and may analyze a vulnerable part of the body and possible diseases according to the inborn body characteristics. The server 10 may predict the onset of possible diseases from the result of inborn body characteristic analysis.

According to an embodiment, the server 10 may collect medical information corresponding to the health analysis result. The medical information may include information on a medical institution that handles the disease (e.g., information collected from a medical institution terminal 30 to be described below), and information on treatment and/or prevention of the disease (e.g., information on food, diet, exercise, and medication), for example, in relation to possible diseases according to the health analysis result.

The server 10 may transmit the aforementioned health analysis result to the user terminal 20. The server 10 may also provide the medical information that is collected in response to the health analysis result, to the user terminal 20. The server 10 may store and manage the health analysis result in a memory or the like.

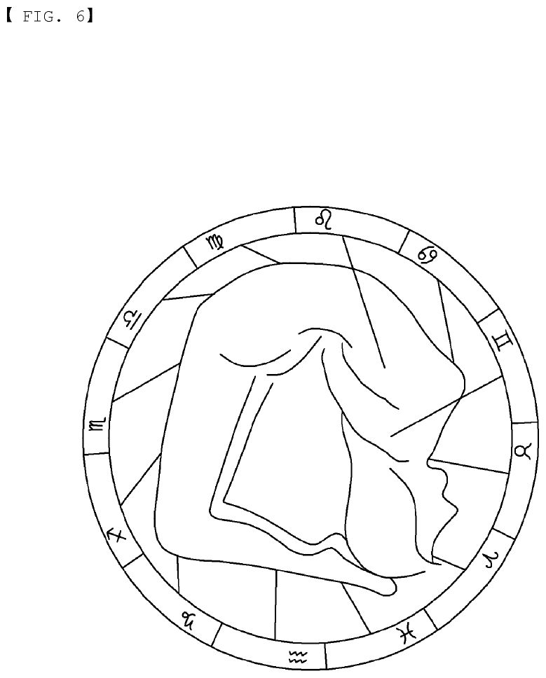

A health analysis method of the server 10 will be described below in more detail with reference to FIGS. 5 to 8.

The user terminal 20 may include, for example, at least one of a smartphone, a tablet PC, a mobile phone, a video phone, a desktop PC, a laptop PC, a netbook computer, a workstation, a PDA, a portable multimedia player (PMP), an MP3 player, a medical device, or a wearable device. The wearable device may include an accessory (e.g., a watch, a ring, a bracelet, an anklet, a necklace, eyeglasses, a contact lens, or a head-mounted-device (HMD)), a textile or garment-integrated device (e.g., electronic garments), a body-attached device (e.g., skin pads or tattoos), or a bioimplantable circuit. In some embodiments, the electronic device may include at least one of a television, a digital video disc (DVD) player, an audio system, a refrigerator, a set-top box, a home automation control panel, or a media box.

The server 10 and the user terminal 20 may be connected to each other through a network 100. According to various embodiments, the network 100 may be a telecommunications network. The telecommunications network may include at least one of a computer network, the Internet, the Internet of things, or a telephone network. Alternatively, the network 100 may include at least one of a telecommunications network, for example, a computer network (e.g., LAN or WAN), the Internet, or a telephone network.

The user terminal 20 may transmit the request signal to use the health analysis service according to the present invention to the server 10. The user terminal 20 may collect user information for requesting the server 10 to perform health analysis and may transmit the collected user information to the server 10.

The user information may include, for example, the date of birth, a birth address, latitude and longitude of the birth address, genetic test record, etc. of the user. According to an embodiment, the user terminal 20 may receive at least some or all of the user information from the user. Alternatively, the user terminal 20 may receive at least some or all of the user information from an external electronic device (e.g., another server or the medical institution terminal 30 to be described below). For example, the user terminal 20 may transmit information on the birth address received from the user to another external server and may receive information on the latitude and longitude of the birth address from the external server. For example, the user terminal 20 may receive information on the genetic test record of the user from another external server.

The user terminal 20 may receive the health analysis result from the server 10. The user terminal 20 may also receive the medical information from the server 10. The user terminal 20 may output the received health analysis result and/or medical information through a display or the like.

Upon receiving an additional request signal about the health analysis result from the user, the user terminal 20 may perform processing accordingly. For example, the user terminal 20 may output the medical institution information of the medical institution selected by the user, may attempt to make a call, to transmit a text message, or to transmit an email to the corresponding medical institution, or may transmit the health analysis result to the corresponding medical institution.

According to various embodiments, some or all of operations executed by the user terminal 20 may be executed by the server 10. For example, when the user terminal 20 needs to perform a predetermined function or service automatically or in response to a request, instead of or in addition to autonomous execution of a function or a service, the user terminal 20 may make a request to the server 10 for at least one some functions related thereto. The server 10 may execute the requested function or an additional function and may transfer the result to the user terminal 20. The user terminal 20 may provide the requested function or service by processing the received result as it is or additionally. To this end, for example, cloud computing, distributed computing, or client-server computing technologies may be used.

For example, the user terminal 20 may transmit information on the birth address of the user to the server 10, the server 10 may transfer the information on the birth address of the user to another external server, and may receive information on the latitude and longitude of the birth address from another server. In addition, for example, the user terminal 20 may make a request to another external server for a genetic test record of the user and another external server may transmit information on the genetic test record of the user to the server 10.

According to various embodiments of the present invention, the health analysis service provision system 1 may further include the medical institution terminal 30. The medical institution terminal 30 may be the same type as the user terminal 20 or a different type therefrom.

The server 10 may receive information on the corresponding medical institution, for example, a name, geographic information, medical staff information, and information on treatment/diagnosis/treatment performance from the medical institution terminal 30. The server 10 may provide the medical institution information collected from the medical institution terminal 30 to the user terminal 20.

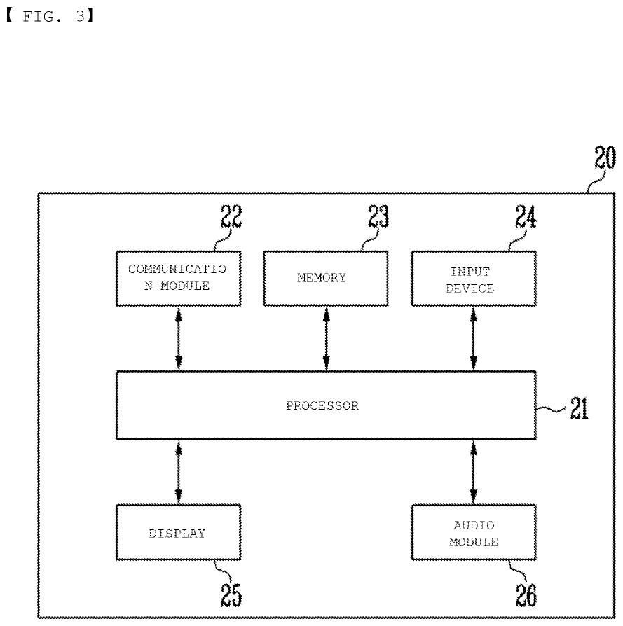

FIG. 2 is a block diagram showing the configuration of a server according to an embodiment of the present invention.

Referring to FIG. 2, the server 10 according to an embodiment of the present invention may include a communication circuit 11, a memory 12, and a processor 13. FIG. 2 shows only components related to various embodiments of the present invention, and needless to say, other components may be provided in addition to the aforementioned components.

The communication circuit 11 may establish a communication connection with at least one external electronic device (e.g., the user terminal 20 and the medical institution terminal 30 of FIG. 1). To this end, the communication circuit 11 may be connected to the network 100 of FIG. 1 and may communicate with the external electronic device through wireless communication or wired communication.

The wireless communication may include, for example, at least one of Wi-Fi, Wi-Fi direct, Bluetooth (BT), near field communication (NFC), global positioning system (GPS), or cellular communication (e.g., LTE, LTE-A, CDMA, WCDMA, UMTS, WiBro, or GSM). The wired communication may include, for example, at least one of universal serial bus (USB), high definition multimedia interface (HDMI), recommended standard 232 (RS-232), or plain old telephone service (POTS).

The communication circuit 11 may be referred to as a communication unit or a communication module, or may include the communication unit or a communication module as a part thereof or may constitute the communication unit or the communication module.

The memory 12 may store a command or data related to at least one other component of the server 10.

According to various embodiments of the present invention, the memory 12 may store terminal information and/or user information of the user terminal 20 of FIG. 1. The terminal information and/or user information of the user terminal 20 may be stored in a database in the memory 12.

The processor 13 may have overall control over the server 10. For example, the processor 13 may acquire the terminal information and/or user information of the user terminal 20, which transfers the request signal to use the health analysis service, and may store and manage acquired information in the memory 12. The processor 13 may perform health analysis on the user of the corresponding user terminal 20 based on the user information, may generate a health analysis result and medical information corresponding thereto, and may transfer the generated information to the user terminal 20.

The processor 13 may store and manage the medical institution information received from the medical institution terminal 30 in the memory 12. When performing health analysis on an arbitrary user, the processor 13 may extract medical institution information corresponding to the health analysis result from the memory 12 and may transmit the extracted information to the user terminal 20 of the corresponding user.

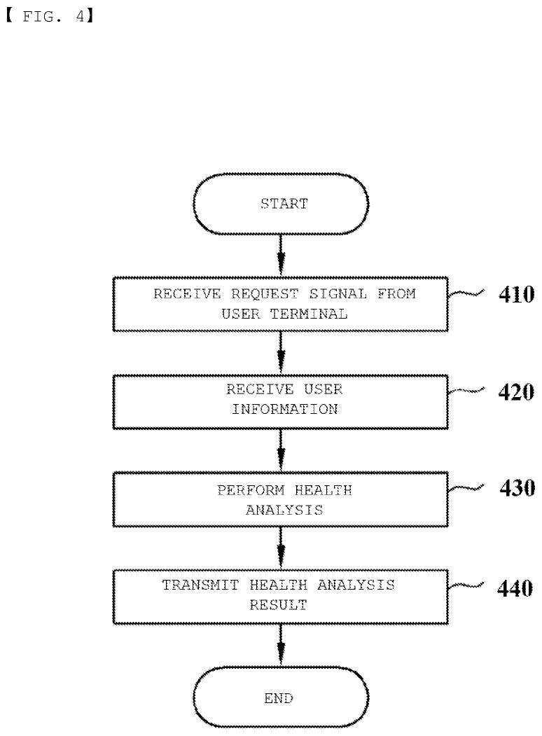

FIG. 3 is a block diagram showing the configuration of a user terminal according to an embodiment of the present invention. Although FIG. 3 exemplifies the user terminal 20 shown in FIG. 1 to describe the present invention, the configuration of the user terminal 20 shown in FIG. 3 may also be applied to the medical institution terminal 30 in the same way.

Referring to FIG. 3, the user terminal 20 may include at least one processor (e.g., AP) 21, a communication module 22, a memory 23, an input device 24, a display 25, and an audio module 26. FIG. 3 shows only components related to various embodiments of the present invention, and needless to say, other components may be provided in addition to the aforementioned components.

The processor 21 may control a plurality of hardware or software components connected to the processor 21 by, for example, driving an operating system or an application program and may process and calculate various pieces of data. The operating system may include, for example, Android™, iOS™, Windows™, Symbian™, Tizen™, or Bada™.

The processor 21 may be embodied in the form of, for example, a system on chip (SoC). According to an embodiment, the processor 21 may further include a graphics processing unit (GPU) and/or an image signal processor. The processor 21 may load the command or data received from at least one of other components (e.g., a non-volatile memory), may process the same, and may store the result data in a non-volatile memory.

The communication module 22 may set communication between the user terminal 20 and an external device (e.g., the server 10 and the medical institution terminal 30). For example, the communication module 22 may be connected to the network 100 and may communicate with an external device through wireless communication or wired communication.

The wireless communication may include cellular communication using at least one of, for example, LTE, LTE advance (LTE-A), code division multiple access (CDMA), wideband CDMA (WCDMA), universal mobile telecommunications system (UMTS), wireless broadband (WiBro), or global system for mobile communications (GSM). According to an embodiment, the wireless communication may include at least one of, for example, Wi-Fi, Bluetooth, Bluetooth low energy (BLE), Zigbee, near field communication (NFC), magnetic secure transmission, radio frequency (RF), or body area network (BAN). According to an embodiment, the wireless communication may include GNSS. The GNSS may be, for example, a global positioning system (GPS), a global navigation satellite system (Glonass), the Beidou Navigation Satellite System (hereinafter “Beidou”), or Galileo, the European global satellite-based navigation system. Hereinafter, in the specification, “GPS” may be used interchangeably with “GNSS”. The wired communication may include at least one of, for example, universal serial bus (USB), high definition multimedia interface (HDMI), recommended standard232 (RS-232), power line communication, or plain old telephone service (POTS).

The memory 23 may store a command or data related to at least one other component of the user terminal 20. In addition, the memory 23 may store software and/or a program. The program may include, for example, kernel, middleware, an application programming interface (API), and/or an application program (or application). At least some of the kernel, the middleware, or the API may be referred to as an operating system. The application may include, for example, a health management application to use the health analysis service according to the present invention.

The memory 23 may include at least one of, for example, a volatile memory (e.g., DRAM, SRAM, or SDRAM), or a non-volatile memory (e.g., one time programmable ROM (OTPROM), PROM, EPROM, EEPROM, mask ROM, flash ROM, flash memory, hard drive, or solid state drive (SSD)).

The input device 24 may include, for example, a touch panel, a (digital) pen sensor, a key, or an ultrasonic input device. The touch panel may use at least one of, for example, an electrostatic method, a resistive method, an infrared method, or an ultrasonic method. The touch panel may further include a control circuit. The touch panel may further include a tactile layer and may provide a tactile response to the user. The (digital) pen sensor may be, for example, a portion of the touch panel or may include a separate recognition sheet. The key may include, for example, a physical button, an optical key, or a keypad. The ultrasonic input device may detect ultrasonic waves generated by an input device through a microphone and may check data corresponding to the detected ultrasonic waves.

According to various embodiments of the present invention, the input device 24 may receive request input to use the health analysis service from the user. In addition, the input device 24 may receive user information requested to use the health analysis service. The input device 24 may receive an additional request of the user for the health analysis result, for example, input of selection of an arbitrary medical institution, and call, text, or email to an arbitrary medical institution.

The display 25 may include, for example, a liquid crystal display (LCD), a light emitting diode (LED) display, an organic light emitting diode (OLED) display, a microelectromechanical system (MEMS) display, or an electronic paper display. The display 25 may display, for example, various content (e.g., text, images, video, icons, and/or symbold) to the user. The display 25 may include a touchscreen, and may receive, for example, touch, gesture, proximity, or hovering input using an electronic pen or a portion of the body of the user.

According to various embodiments of the present invention, the display 25 may display the health analysis result received from the server 10. For example, the display 25 may display the onset of disease included in the health analysis result in the form of a special graph, a figure, or a number. Upon receiving the medical information from the server 10, the display 25 may display the medical information. For example, the display 25 may display a list of at least one piece of medical institution information included in the medical information.

The audio module 26 may convert, for example, sound and electrical signals in both directions. The audio module 26 may process input or output sound information through, for example, a speaker, a receiver, an earphone, or a microphone.

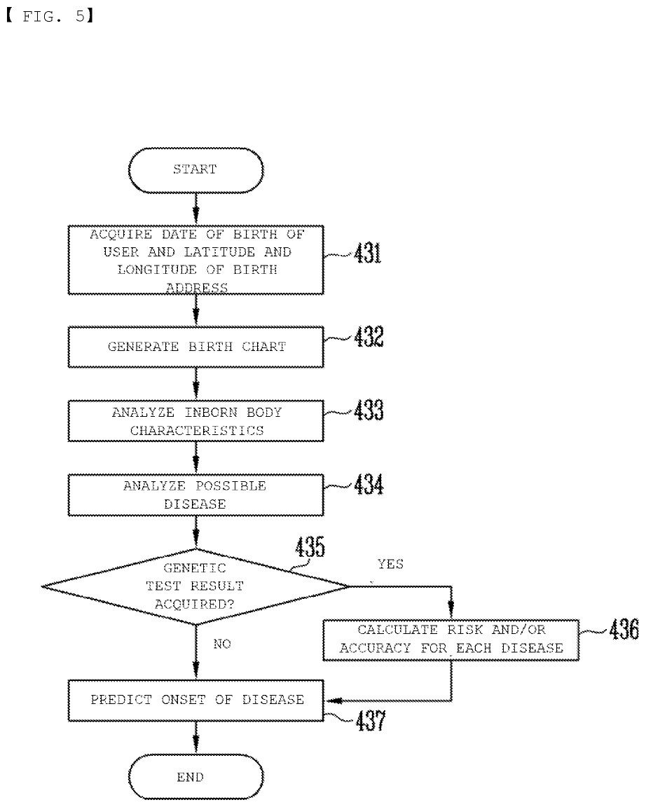

FIG. 4 is a flowchart of a method of providing a health analysis service of a server according to an embodiment of the present invention.

First, the server 10 may receive a request signal to use a health analysis service from the user terminal 20 (410). The request signal may be transmitted to the server 10, for example, in response to user input generated by the user terminal 20.

The server 10 may store the terminal information of the user terminal 20 in response to the request signal. According to an embodiment, the terminal information may include identification information (e.g., device identification (ID)) of the corresponding user terminal 20, and network address information (e.g., a media access control (MAC) address). Storage of the terminal information is not necessarily required in the present invention, and when registration and authentication of the user terminal 20 is not required to use the health analysis service, this process may be omitted.

According to an embodiment, when user information is also included in the received request signal, the server 10 may store the user information. When the user information is not included in the received request signal, the server 10 may additionally receive the user information from the user terminal 20 (420). The user information may be received from the user terminal 20 in response to a request of the server 10. Upon receiving the user information from the user terminal 20, the server 10 may store the received user information in association with the terminal information of the user terminal 20. According to an embodiment, the user information may include the date of birth, birth address, genetic test record, etc. of the user of the corresponding user terminal 20.

Then, the server 10 may perform health analysis on the corresponding user based on the collected user information (430). According to various embodiments of the present invention, the server 10 may perform health analysis based on the inborn body characteristics of the user. In detail, the server 10 may generate a birth chart (or inborn body characteristic table or zodiac signs (horoscope)) indicating the positions of the celestial bodies in the solar system on the zodiac signs at the time of birth of the user based on the date of birth of the user and the latitude and longitude of the place of birth. The server 10 may detect planets arranged at a special angle based on the center of the zodiac signs among the celestial bodies of the solar system. The server 10 may determine the body part corresponding to the zodiac signs in which the detected planets are arranged, as a vulnerable part. In contrast, the server 10 may detect planets arranged at another special angle and may determine the body part corresponding to the zodiac signs in which the detected planets are arranged, as a healthy part. The server 10 may determine possible diseases related to the determined vulnerable part.

According to various embodiments of the present invention, the server 10 may predict the onset of possible diseases. For example, the server 10 may determine the time of placing a celestial body, which is a cause of determination of the corresponding disease, at the same angle as in the birth chart of the user using ephemeris and may predict the corresponding time as the onset of the disease.

A health analysis method of the server 10 will be described below in more detail with reference to FIGS. 5 to 8.

The server 10 may transmit the health analysis result as generated above to the corresponding user terminal 20 (440).

According to an additional embodiment of the present invention, the server 10 may collect and store medical institution information from at least one medical institution terminal 30. According to the embodiment, the server 10 may collect the medical institution information of medical institutions corresponding to the health analysis result. For example, the server 10 may collect the health analysis result and the medical institution information of the medical institution that handles disease determined to be a possible disease.

The server 10 may collect the health analysis result and information on treatment and/or prevention of disease determined to be a possible disease. The information on treatment and/or prevention may include, for example, information on food, diet, exercise, and medication.

The server 10 may additionally transmit the medical information including the collected medical institution information and the information on treatment and/or prevention to the user terminal 20.

An additional request signal may be received from the user terminal 20. The request signal may be related to information on health analysis. For example, the request signal may include a request for transmitting the information on health analysis to an arbitrary medical institution terminal 30 or another external electronic device.

The request signal may be related to medical information. For example, the request signal may include a request for selecting an arbitrary medical institution included in the medical institution information or call, text, or email the selected medical institution. For example, the request signal may include a request for transmitting the information on health analysis to the selected medical institution.

As described above, according to the present invention, health of the user may be analyzed based on the inborn body characteristics determined depending on the date of birth and birth address of the user, and thus useful health analysis results customized for each individual may be provided. As described above, according to the present invention, the health analysis result of the user may be provided to a medical institution or the like, or information on treatment and/or prevention of possible diseases may be provided to the user, and thus the user may easily manage his or her health. As such, the present invention may stimulate interest in health of the user and may contribute to development of advanced medical industry using telecommunication.

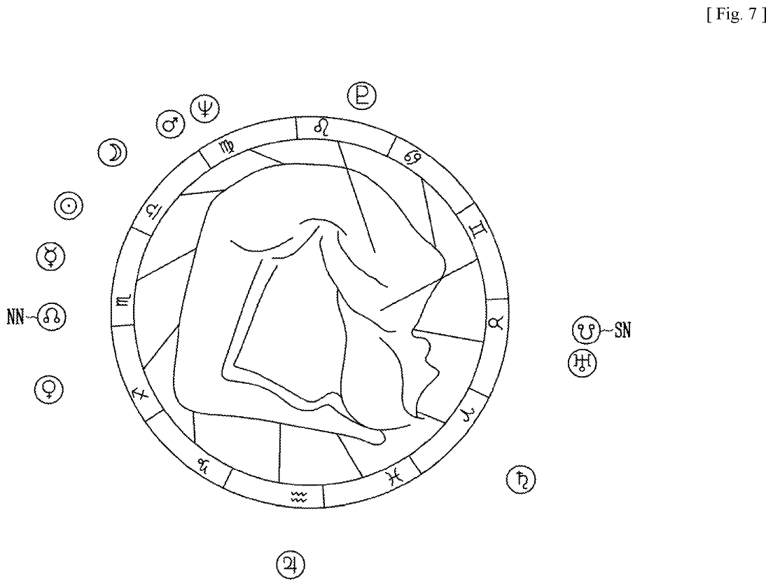

FIG. 5 is a flowchart of a health analysis method of a server according to an embodiment of the present invention. FIGS. 6 to 8 are diagrams for explaining a health analysis method according to an embodiment of the present invention.

According to various embodiments of the present invention, the health analysis method of FIGS. 6 to 8 may be performed by the server 10.

First, the server 10 may acquire the date of birth of the user and the latitude and longitude of a birth address (431). According to an embodiment, the date of birth and the latitude and longitude of the birth address may be acquired from the user information received from the user terminal 20. According to an embodiment, upon receiving only information on the birth address from the user terminal 20, the server 10 may acquire information on the latitude and longitude of the birth address from another external server, or the like.

Then, the server 10 may generate a birth chart of the user based on the date of birth of the user and the latitude and longitude of the birth address (432). The birth chart may refer to an inborn body characteristic table or zodiac signs, which will be described below in detail.

The zodiac signs may mean 12 major constellations placed in respective areas when a circular path along which the sun performs annual motion around the earth, that is, the ecliptic, is divided into 12 parts. Here, the 12 constellations may include Aries, Taurus, Gemini, Cancer, Leo, Virgo, Libra, Scorpio, Sagittarius, Capricorn, Aquarius, and Pisces. The 12 constellations may be represented by unique symbols as shown in Table 1 below.

TABLE 1

| | |
|---|---|
| [symbol] | (Aries) |
| [symbol] | (Taurus) |
| [symbol] | (Gemini) |
| [symbol] | (Cancer) |
| [symbol] | (Leo) |
| [symbol] | (Virgo) |
| [symbol] | (Libra) |
| [symbol] | (Scorpio) |
| [symbol] | (Sagittarius) |
| [symbol] | (Capricorn) |
| [symbol] | (Aquarius) |
| [symbol] | (Pisces) |

Main planets (hereinafter planets) that make up the solar system with the Earth, that is, the sun, the Moon, Mercury, Venus, Mars, Jupiter, Saturn, Uranus, Neptune, and Pluto may move along the ecliptic, and may be positioned in a region in which an arbitrary constellation is positioned at an arbitrary point in time. The main planets may be represented by unique symbols as shown in Table 2 below.

TABLE 2

| | |
|---|---|
| [symbol] | (Sun) |
| [symbol] | (Luna) |
| [symbol] | (Mercury) |
| [symbol] | (Venus) |
| [symbol] | (Mars) |
| [symbol] | (Jupiter) |
| [symbol] | (Saturn) |
| [symbol] | (Uranus) |
| [symbol] | (Neptune) |
| [symbol] | (Pluto) |

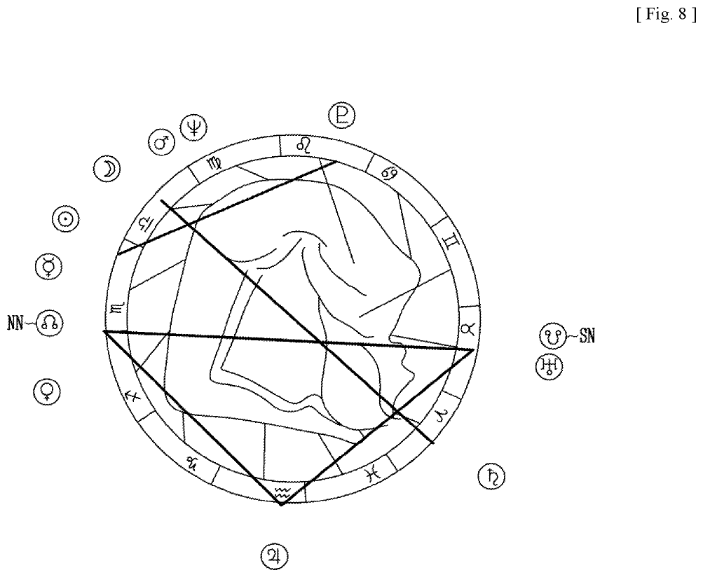

The server 10 may determine the positions of planets arranged on the zodiac signs based on the date of birth of the user and the place of birth. Arrangement of planets of the zodiac signs may be referred to as a birth chart, an inborn body characteristic table, or zodiac signs. An embodiment of the birth chart is shown in FIG. 7. The server 10 may analyze the inborn body characteristics of the user based on the generated birth chart (433).

Each of the zodiac signs may correspond to parts of the body as shown in FIGS. 6 to 8. According to an embodiment, the parts of the body corresponding to the zodiac signs may be defined as shown in Table 3 below.

TABLE 3

| | |
|---|---|
| Aries | Head, cerebral hemisphere, skull, eye, face, |
| | upper jaw, internal carotid artery, iron in |
| | blood |
| Taurus | Neck, tongue, roof of mouth, throat, occipital |
| | region, tonsils, mandible, ear, cerebellum, |
| | external carotid artery, carotid vein, thyroid |
| | gland |
| Gemini | Shoulders, arms, hands, lungs, upper ribs, |
| | bronchi, capillaries, breathing |
| Cancer | Stomach, esophagus, diaphragm, chest, breast, |
| | upper liver, pancreas, serum, lymph |
| Leo | heart (inflammation), spine, aorta |
| Virgo | Abdomen, large intestine, small intestine, lower |
| | liver lobe, spleen, duodenum |
| Libra | Kidney, adrenal gland, lumbar (lower back), |
| | skin, vasomotor nerve |
| Scorpio | Bladder, urethra, genitals, descending colon |
| | (part of large intestine), sigmoid colon, |
| | prostate, nose, and elements that make the blood |
| | red |
| Sagittarius | Hip, thigh, ileum (part of small intestine), |
| | coccyx, sacrum (nerve), sciatic bone (nerve) |
| Capricorn | Bones, knees, joints, hair |
| Aquarius | Shin, ankle |
| Pisces | Feet, toes, and fibrin in blood |

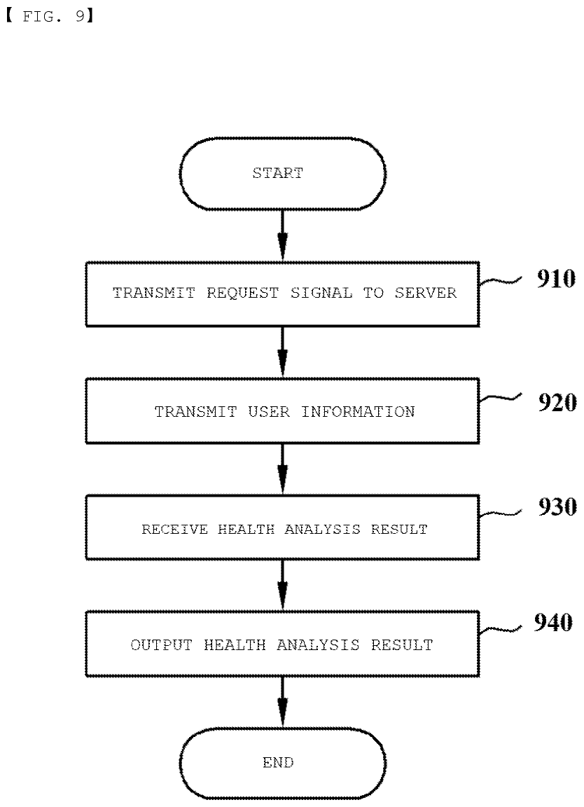

The server 10 may detect planets arranged at a preset angle based on the center of the zodiac signs. Here, the preset angle may include, for example, at least one of 45 degrees, 90 degrees, or 180 degrees. For example, in the embodiment shown in FIG. 8, the Moon and Saturn may be arranged at 180 degrees to each other based on the center of the zodiac signs. In the embodiment shown in FIG. 8, Pluto and Mercury may be arranged at 90 degrees to each other based on the center of the zodiac signs. In the embodiment shown in FIG. 8, Uranus and Jupiter may be arranged at 45 degrees to each other based on the center of the zodiac signs (an acute angle of a right triangle is 45 degrees). The server 10 may detect planets arranged at a preset angle based on the center of the zodiac signs using the method.

The server 10 may determine body parts corresponding to the zodiac signs in which the detected planets are arranged. For example, in the embodiment shown in FIG. 8, the server 10 may determine body parts corresponding to Libra and Aries, in which the Moon and Saturn are respectively arranged, (e.g., kidney, adrenal gland, lumbar (lower back), skin, vasomotor nerve, head, cerebral hemisphere, skull, eye, face, upper jaw, internal carotid artery, and iron in blood) as vulnerable parts.

According to an embodiment, the server 10 may assign weights to the vulnerable parts depending on the number of detected planets with respect to each of the zodiac signs. For example, when a larger number of planets is detected for an arbitrary constellation, the server 10 may determine the body part corresponding to the corresponding constellation to be more vulnerable.

The determination result in that a specific part of the body is vulnerable may be the inborn body characteristics of the user.

According to an embodiment, the server 10 may additionally determine a healthy part of the body. For example, the server 10 may detect planets arranged at a preset different angle based on the center of the zodiac signs. Here, the preset different angle may include, for example, at least one of 30 degrees, 60 degrees, or 120 degrees. The server 10 may determine body parts corresponding to the zodiac signs in which the detected planets are arranged, as healthy parts.

The server 10 may analyze possible diseases in response to the determined vulnerable part (434). The server 10 may determine a related disease as a possible disease based on a medical database with respect to the body part determined as the vulnerable part.

According to an embodiment of the present invention, the server 10 may determine the risk and/or accuracy for the possible diseases. The risk and/or the accuracy may be determined based on the genetic test result of the user. However, the technical spirit of the present invention is not limited thereto, and the risk and/or the accuracy may be determined based on the user information or various pieces of separately collected medical information.

In detail, the server 10 may determine whether the genetic test result for the user is acquired (435). The genetic test result may be received as user information from the user terminal 20 or may be acquired from another external server associated with the medical institution terminal 30.

When the genetic test result is not acquired, the server 10 may perform a subsequent operation without determination of the risk and/or the accuracy. When the genetic test result is acquired, the server 10 may calculate the risk and/or the accuracy for each disease (436).

In detail, the server 10 may determine whether the possible disease determined based on the inborn body characteristics is included in the disease list determined based on the genetic test result. When the possible disease determined based on the inborn body characteristics is included in the disease list determined based on the genetic test result, the server 10 may relatively increase the risk and/or the accuracy of the corresponding disease. In contrast, when the possible disease determined based on the inborn body characteristics is not included in the disease list determined based on the genetic test result, the server 10 may relatively reduce the risk and/or the accuracy of the corresponding disease.

The server 10 may predict the onset of the determined disease (437). The server 10 may determine the time of placing a planet, which is a cause of determination of the disease, at the same angle as in the birth chart depending on an orbital period. To this end, the server 10 may use ephemeris. For example, in the embodiment of FIG. 8, the server 10 may determine the time of positioning the Moon and Saturn at 180 degrees, 90 degrees, and 45 degrees based on the center of zodiac signs from the ephemeris. The server may predict that the possible diseases determined in response to the Moon and Saturn occur at the time determined using the ephemeris.

According to an embodiment, the server 10 may determine the likelihood (probability) of the disease based on similarity between a relative angle of planets, which correspond to a cause of determination of the disease, due to an orbital period and an angle at which the planets are arranged in a birth chart. In the embodiment, the likelihood at an arbitrary time may be calculated using “arranged angle at arbitrary time/arranged angle in birth chart×100”.

For example, in the embodiment of FIG. 8, the server 10 may determine the likelihood of the related disease at the time when the Moon and Saturn are arranged at 180 degrees to each other based on the center of the zodiac signs as 100% and may determine the likelihood of the related disease at the time when the Moon and Saturn are arranged at 90 degrees to each other based on the center of the zodiac signs as 50%, from ephemeris.

According to various embodiments of the present invention, the server 10 may further determine the characteristics of the user as well as the aforementioned health analysis. The characteristics of the user may include, for example, personality, disposition, fate, fortune, cause of disease, life goal, life roadmap, etc.

For example, the server 10 may determine a constellation in which the sun, the Moon, and a south node (SN), which is a southern intersection between the ecliptic and the equator, are placed. The server 10 can determine the individuality, dominant emotion and sentiment of the user, a method of expressing the inner, etc. of the user based on a predetermined keyword for the determined constellation. The server 10 may further determine a cause of the disease from the information.

The server 10 may determine a constellation in which a south node (SN), which is a southern intersection between the ecliptic and the equator, and a north node (NN), which is a northern intersection between the ecliptic and the equator, are placed. The server 10 may determine karma of the user and the cause of the disease based on the predetermined keyword for the determined constellation. Here, the keyword may include, for example, freedom, introspection, responsibility, influence, reform, and artistry.

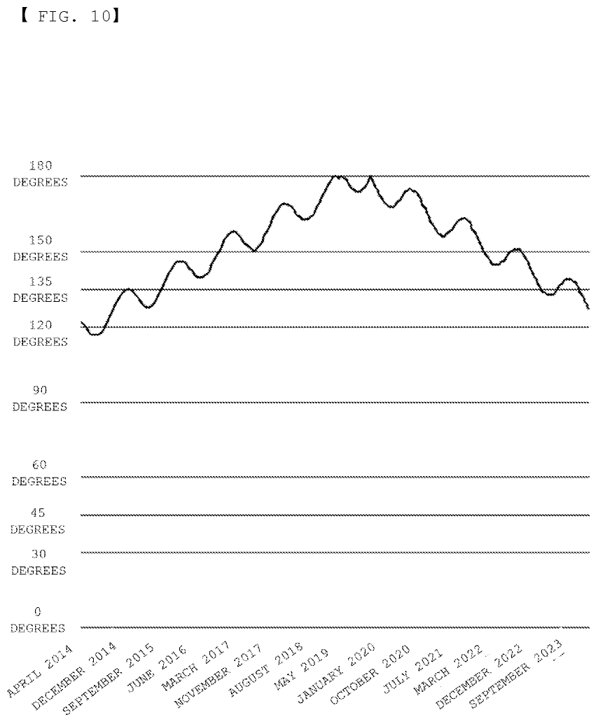

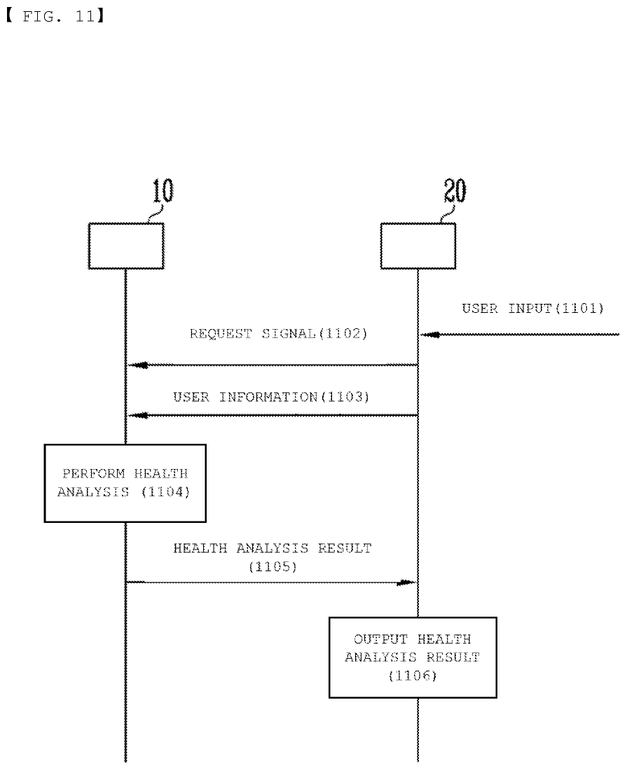

FIG. 9 is a flowchart of a method of providing a health analysis service of a user terminal according to an embodiment of the present invention. FIG. 10 is a diagram showing an image of a health analysis result output on a user terminal according to an embodiment of the present invention.

Referring to FIG. 9, first, the user terminal 20 may transmit a request signal to use the health analysis service according to the present invention to the server 10 (910). The request signal may be generated in response to, for example, user input received from the user terminal 20.

According to an embodiment, the request signal may include identification information (e.g., device identification (ID)) of the corresponding user terminal 20, and network address information (e.g., a media access control (MAC) address) as terminal information. According to an embodiment, the request signal may include the date of birth of the user of the user terminal 20, a birth address, and genetic test record as user information.

When the user information is not included in the request signal, the user terminal 20 may further transmit the user information to the server 10 (920). According to an embodiment, the request for the user information may be performed in response to a request of the server 10. According to various embodiments, the user information may include, for example, at least one of the date of birth of the user, a birth address, latitude and longitude of the birth address, or genetic test result.

When health analysis is completely performed based on the user information transmitted to the server 10, the user terminal 20 may receive the health analysis result from a server (930). The health analysis result may include, for example, a vulnerable part, a possible disease, and the onset of possible diseases based on the inborn body characteristics of the user. In some embodiments, the health analysis result may further include the characteristics of the user, which are further determined by the server 10.

The user terminal 20 may output the received health analysis result (940). The health analysis result may be output through the display 25 and/or the audio module 26. According to an embodiment, the vulnerable part of the body may be output with the weight determined by the server 10 or may be aligned based on the weight and may be output. According to an embodiment, the possible disease may be output with the risk and/or the accuracy determined by the server 10 or may be aligned based on the risk and/or the accuracy and may be output.

According to an embodiment, the onset of the disease included in the health analysis result may be output in the form including at least one of a graph, a figure, or a number, indicating the likelihood of the disease over the time shown in FIG. 10. The likelihood of the disease of the graph shown in FIG. 10 may be output as similarity between a relative angle of planets, which correspond to a cause of determination of the disease, due to an orbital period and an angle at which the planets are arranged in a birth chart, as described above.

According to an embodiment, upon receiving medical information from the server 10, the user terminal 20 may output medical information with the health analysis result and/or in response to a request of the user. The medical information may include information on a medical institution that handles the disease (e.g., information collected from a medical institution terminal 30 to be described below), and information on treatment and/or prevention of the disease (e.g., information on food, diet, exercise, and medication), for example, in relation to possible diseases according to the health analysis result.

According to various embodiments of the present invention, the user terminal 20 may receive additional input of the user for the output health analysis result and/or medical information. The additional input may include, for example, a request for selecting any one medical institution of the medical institution information and calling, texting, or emailing to the selected medical institution. In the embodiment, the user terminal 20 may perform a processing operation corresponding to the additional input.

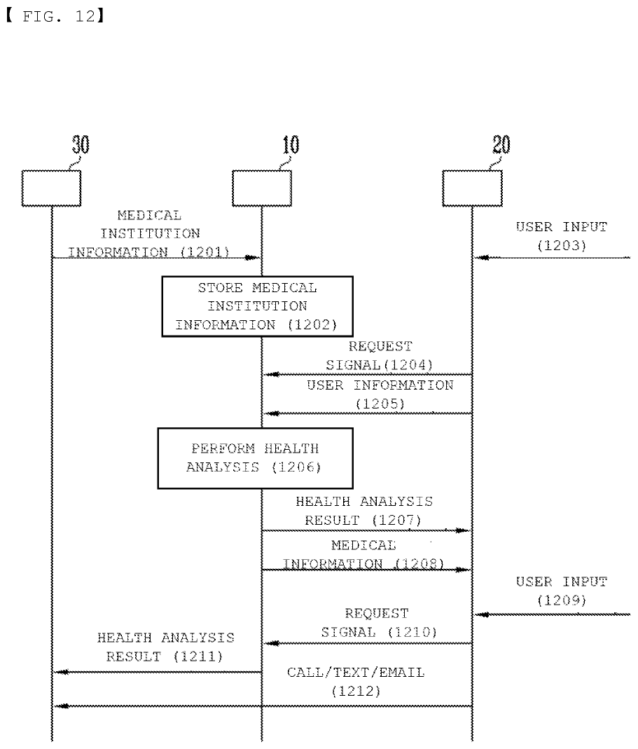

FIG. 11 is a flowchart showing a method of providing a health analysis service according to an embodiment of the present invention.

Referring to FIG. 11, according to an embodiment of the present invention, the user terminal 20 may receive user input to use the health analysis service from the user (1101). The user terminal 20 may transmit the request signal to use the health analysis service to the server 10 in response to the user input (1102). According to an embodiment, when the request signal does not include user information, the user terminal 20 may further transmit the user information to the server 10 (1103). The user information may be transmitted when a separate request is received from the server 10. For example, upon receiving the request for the user information from the server 10, the user terminal 20 may output an input window of the user information, may receive the user information from the user, and may transmit the user information to the server 10.

When collecting the user information, the server 10 may perform health analysis (1104). The health analysis according to the present invention may be customized to the user based on the inborn body characteristics described with reference to FIG. 5.

When health analysis is completed, the server 10 may transmit the health analysis result to the user terminal 20 (1105). According to an embodiment, the server 10 may transmit the medical information with the health analysis result to the user terminal 20.

The user terminal 20 may output the health analysis result received from the server 10 in the form including at least one of a graph, a figure, or a number. Upon receiving the medical information from the server 10, the user terminal 20 may further output the medical information with the health analysis result or in response to a user request.

FIG. 12 is a flowchart showing a method of providing a health analysis service according to another embodiment of the present invention.

Referring to FIG. 12, according to another embodiment of the present invention, the server 10 may receive medical institution information from at least one medical institution terminal 30 (1201). The server 10 may store and manage the medical institution information (1202).

Then, similarly to FIG. 11, upon receiving the user information from the user terminal 20, the server 10 may transmit the health analysis result to the user terminal 20 based on the user information (1203 to 1207). In this case, the server 10 may further transmit the medical information to the user terminal 20 in response to the health analysis result (1208). The medical information may include medical institution information collected from the medical institution terminal 30.

Then, upon receiving user input corresponding to the medical information (1209), the user terminal 20 may transmit the request signal corresponding to the user input to the server 10 (1210). Here, the request signal may be for requesting the server 10 to transmit the health analysis result to a specific medical institution terminal 30. In the embodiment, the server 10 may transmit the health analysis result for the corresponding user to the corresponding medical institution terminal 30 (1211).

The user input may be for call, text, or email to an arbitrary medical institution. In the embodiment, the user terminal 20 may attempt direct call to the medical institution terminal 30 or may transmit a text, and/or mail based on the medical institution information of the corresponding medical institution (1212).

One of ordinary skill in the art to which the present invention pertains will be able to make various modifications and variations without departing from the essential characteristics of the present invention. In addition, the embodiments disclosed in the specification and drawings are merely provided for specific examples to easily explain and understand the contents of the present invention and are not intended to limit the scope of the present invention. Accordingly, it should be construed that all changes or modifications derived from the technical spirit of the present invention in addition to the embodiments disclosed herein are included in the scope of the present invention.

1. A health analysis service provision apparatus comprising:
a communication circuit configured to communicate with a user terminal; and
a processor configured to perform health analysis based on user information received through the communication circuit and to control the communication circuit to transmit a health analysis result to the user terminal,
wherein:
the processor analyzes inborn body characteristics of a user using a date of birth of the user and a birth address, included in the user information, predicts a possible disease and an onset of the possible disease based on the inborn body characteristics, generates zodiac signs at a time of birth of the user based on latitude and longitude at the time of birth of the user, generates a birth chart indicating positions of celestial bodies of a solar system on the zodiac signs, detects a planet positioned at at least one preset angle based on a center of the zodiac signs of the celestial bodies of the solar system, determines a body part corresponding to the zodiac signs in which the detected planet is placed, as a vulnerable part, to determine the inborn body characteristics, and determines the possible disease based on the vulnerable part of the inborn body characteristics;
the communication circuit transmits the inborn body characteristics and the possible disease as health analysis information to the user terminal, collects information on a medical institution related to the possible disease included in the health analysis information, receives information on an arbitrary medical institution selected by the user from the user terminal, and transmits information on a medical institution related to the possible disease included in the health analysis information, to the user terminal, or transmits the information on the medical institution related to the possible disease to the arbitrary medical institution that receives the health analysis information from the user terminal;
the processor corrects risk or accuracy for each disease based on a genetic test result of the user, received through the communication circuit, compared with the possible disease determined based on the inborn body characteristics; and
the communication circuit transmits the corrected risk or accuracy for each disease to the user terminal.

2. The health analysis service provision apparatus according to claim 1, wherein the at least one preset angle includes at least one of 45 degrees, 90 degrees, and 180 degrees.

3. The health analysis service provision apparatus according to claim 1, wherein the processor determines a time when the at least one detected planet is placed in zodiac signs at a time of birth based on ephemeris, and determines the determined time as the onset of the possible disease.

4. The health analysis service provision apparatus according to claim 3, wherein the processor determines likelihood of the possible disease based on similarity between an angle of the at least one planet detected at an arbitrary time based on the zodiac signs and an angle of the planet placed in the zodiac signs at the time of birth.

5. The health analysis service provision apparatus according to claim 4, wherein information on the likelihood over time is transmitted to the user terminal to be output in a form including at least one of a graph, a figure, or a number by the user terminal.

6. The health analysis service provision apparatus according to claim 1, wherein the processor detects at least one different planet placed at another preset different angle based on the center of the zodiac signs and determines a body part corresponding to a constellation in which the at least one detected different planet is placed, as a healthy part of the user.

7. The health analysis service provision apparatus according to claim 6, wherein the at least one preset different angle includes at least one of 30 degrees, 60 degrees, or 120 degrees.

8. A method of providing a health analysis service of a server including a communication circuit and a processor, the method comprising:
receiving user information from a user terminal by the communication circuit;
analyzing inborn body characteristics of the user using a date of birth of the user and a birth address, included in the user information, by the processor; and
predicting a possible disease and an onset of the possible disease based on the inborn body characteristics, by the processor,
wherein the analyzing includes:
generating zodiac signs at a time of birth of the user based on latitude and longitude at the time of birth of the user;
generating a birth chart indicating positions of celestial bodies of a solar system on the zodiac signs;
detecting a planet positioned at at least one preset angle based on a center of the zodiac signs of the celestial bodies of the solar system; and
determining a body part corresponding to the zodiac signs in which the detected planet is placed, as a vulnerable part, to determine the inborn body characteristics;
wherein the predicting includes:
determining the possible disease based on the vulnerable part of the inborn body characteristics;
transmitting the inborn body characteristics and the possible disease as health analysis information to the user terminal, by the communication circuit;
collecting information on a medical institution related to the possible disease included in the health analysis information, by the communication circuit;
receiving information on an arbitrary medical institution selected by the user from the user terminal; and
transmitting information on a medical institution related to the possible disease included in the health analysis information, to the user terminal, or transmitting the information on the medical institution related to the possible disease to the arbitrary medical institution that receives the health analysis information from the user terminal; and
wherein the method further includes:
correcting risk or accuracy for each disease based on a genetic test result of the user, received through the communication circuit, compared with the possible disease determined based on the inborn body characteristics, by the processor; and
transmitting the corrected risk or accuracy for each disease to the user terminal, by the communication circuit.

9. The method according to claim 8, wherein the predicting includes:
determining the possible disease based on the vulnerable part, by the processor;
determining a time when the at least one detected planet is placed in zodiac signs at a time of birth based on ephemeris, by the processor; and
determining the determined time as the onset of the possible disease.

10. The method according to claim 9, wherein the predicting includes:
determining likelihood of the possible disease based on similarity between an angle of the at least one planet detected at an arbitrary time based on the zodiac signs and an angle of the planet placed in the zodiac signs at the time of birth.

11. The method according to claim 10, wherein information on the likelihood over time is transmitted to the user terminal to be output in a form including at least one of a graph, a figure, or a number by the user terminal.

12. The method according to claim 8, wherein the analyzing includes:
detecting at least one different planet placed at at another preset different angle based on the center of the zodiac signs, by the processor; and
determining a body part corresponding to a constellation in which the at least one detected different planet is placed, as a healthy part of the user, by the processor.
